(19) United States
(12) Patent Application Publication
(10) Pub. No.: US 2003/0216729 A1
(43) Pub. Date: Nov. 20, 2003

(54) Device and method for wound healing and uses therefor

(76) Inventors: Kevin S. Marchitto, Golden, CO (US); Stephen T. Flock, Arvada, CO (US)

Correspondence Address: Benjamin Aaron Adler, ADLER & ASSOCIATES, 8011 Candle Lane, Houston, TX, 77071, US

(21) Appl. No.: 10/441,341

(22) Filed: May 20, 2003

Related U.S. Application Data
(60) Provisional application No. 60/381,948, filed on May 20, 2002.

(51) Int. Cl. A61B 18/18

(52) U.S. Cl. 606/41

(57) ABSTRACT

Provided herein is a device for treating tissue in an individual to effect a weld between said tissue and at least one substrate comprising a material which functions as a fusion composition between the tissue and the substrate(s); a conducting element; a means of delivering a high frequency voltage or current or radiofrequency energy to the conducting element; and a means to control the extent of weld effected. Also provided are methods using the device.

### CROSS-REFERENCE TO RELATED APPLICATION

This non-provisional application claims benefit of provisional U.S. Serial No. 60/381,948, filed May 20, 2002, now abandoned.

### BACKGROUND OF THE INVENTION

1. Field of the Invention

The present invention relates generally to the fields of biomedical engineering, biochemistry and surgery. More specifically, the present invention provides a device and methods for improving the ease with which tissue can be fused to tissue or other materials, or with which cavities in tissues can be sealed.

2. Description of the Related Art

Effective closure of surgical wounds, including incisions, tears and leaks in the patient's organs is critical to the success of the surgical procedure. This success is based on restoration of the physical integrity and function of injured or diseased tissue. Failure to close surgical wounds optimally can also result in serious and excessive scarring. A variety of devices have been developed to assist the surgeon with surgical closure of tissue, including sutures, staples and fibrin glues.

Historically, wound dressings consist of some type of bandage or adhesive. More recently, wound sealing methods whereby ambient energy is directed to the tissue have been tested and occasionally are used clinically. Traditional techniques of managing the wound include cleansing and debriding, treating with antibiotics and applying a dressing. Modern wound care products often seek to provide moisture, pH balance and nutrition in an effort to improve the potential for healing. The healing process may also complicate the status of the patient through formation of scar tissue. This scarring helps to close the wound, but its formation is accompanied by contraction and buildup of tissue which can lead to a loss in flexibility at the wound site and, in severe cases, may result in loss of mobility to the patient.

Commercial electrosurgery and electrocautery devices commonly are used for sealing internal wounds, such as those arising through surgical intervention. Inventions for sealing vessels using other forms of electromagnetic energy have been published. U.S. Pat. No. 6,033,401 describes a device to deliver adhesive and apply microwave energy to effect sealing of a vessel. U.S. Pat. No. 6,179,834 discloses a vascular sealing device to provide a clamping force while radiofrequency energy is applied until a particular temperature or impedance is reached. U.S. Pat. No. 6,132,429 describes using a radiofrequency device to weld blood vessels closed and monitoring the process by changes in tissue temperature or impedance. Nevertheless, these devices are generally unsuitable for the purpose of occluding a wound thereby enhancing long-term healing.

There has been an effort recently to identify biocompatible molecules which can be used as a “fusion composition”. Biomolecules such as fibrin, elastin, albumin have been or are used to “glue” tissue-to-tissue. A number of patents describe the “activation” of these biomolecules to form “welds” through irradiation, often in the form of laser radiant energy, but sometimes in the form of ultrasound or radiofrequency waves. The applied energy is believed to denature the molecules, which then adhere to one-another, or cross-link upon renaturation thereby effecting a bond.

Over the past fifteen years, a significant amount of scientific research has focused on using laser heated “solder” for “welding” tissues such as blood vessels (1-2). Research has been done on laser tissue welding with albumin solders, which is an improvement over conventional suture closure because it offers an immediate watertight tissue closure, decreased operative time, especially in microsurgical or laparoscopic applications, reduced trauma, and elimination of foreign body reaction to sutures, collagen-based plugs and clips. The procedure has been enhanced with the use of advanced solders, strengthening structures, concurrent cooling, and added growth factors (e.g. U.S. Pat. No. 6,221,068).

Use of lasers for tissue welding appeared very promising, however, the techniques have certain limitations. The laser energy must be manually directed by the surgeon, which leads to operator variability. In addition, the radiant energy is not dispersed evenly through the tissue. The high energy at the focal point may result in local bums, and the heating effect drops off rapidly at a small distance from the focal point. Finally, lasers are expensive, and cannot currently be easily miniaturized.

U.S. Pat. No. 5,669,934 describes a method for joining or restructuring tissue consisting of providing a preformed film or sheet of a collagen and/or gelatin material which fuses to tissue upon the application of continuous inert gas beam radiofrequency energy. Similarly, U.S. Pat. No. 5,569,239 describes laying down a layer of energy reactive adhesive material along the incision and closing the incision by applying energy, either optical or radiofrequency energy, to the adhesive and surrounding tissue. Similarly U.S. Pat. Nos. 5,209,776 and 5,292,362 describe a tissue adhesive that is principally intended to be used in conjunction with laser radiant energy to weld severed tissues and/or prosthetic material together. U.S. Pat. No. 6,110,212 describes the use of elastin and elastin-based materials which are biocompatible and can be used to effect anastomoses and tissue structure sealing upon the application of laser radiant energy. The stated benefits, inter alia, are the biocompatible and ubiquitous nature of elastin.

U.S. Pat. No. 6,302,898 describes a device to deliver a sealant and energy to effect tissue closure. It also discloses pre-treating the tissue with energy in order to make the subsequently applied sealant adhere better. PCT Appplication No. WO 99/65536 describes tissue repair by pre-treating the substantially solid biomolecular solder prior to use. U.S. Pat. No. 5,713,891 discloses the addition of bioactive compounds to the tissue solder in order to enhance the weld strength or to reduce post-procedure hemorrhage.

U.S. Pat. No. 6,221,068 teaches the importance of minimizing thermal damage to the tissue to be welded. The method employs pulsed laser irradiation and allowing the tissue to cool to nearly the initial temperature between each heating cycle. U.S. Pat. No. 6,323,037 describes the addition of an “energy converter” to the solder mixture such that optical energy will be efficiency and preferentially absorbed by the solder which subsequently will effect a tissue weld.

Inductive heating (3) is a non-contact process whereby electrical currents are induced in electrically conductive materials (susceptors) by a time-varying magnetic field. Generally, induction heating is an industrial process often used to weld, harden or braze metal-containing parts in manufacturing where control over the heating process and minimized contact with the workpiece are critical. Basically, radiofrequency power is coupled to a conducting element, such as a coil of wire, which serves to set up a magnetic field of a particular magnitude and spatial extent. The induced currents or Eddy currents flow in the conductive materials in a layer referred to as the skin depth δm), given by:

δ=v(2ρ/μΩ),

where Ω is frequency (rads/s), ρ is resistivity (ohm-m) and μ is the permeability (Webers/amp/m) which is the product of μo the permeability of free space and μr the relative permeability of the material.

The magnetic permeability of a material is quantification of the degree to which it can concentrate magnetic field lines. Note, however, that the permeability is not constant in ferromagnetic substances like iron, but depends on the magentic flux and temperature. The skin depth at room temperature at 1 MHz electromagnetic radiation in copper is 0.066 mm and in 99.9% iron is 0.016 mm.

The consequence of current flowing is Joule, or I²R, heating. The skin-depth formula leads to the conclusion that, with increased frequency, the skin depth becomes smaller. Thus, higher frequencies favor efficient and uniform heating of smaller components. In certain situations localized heat can also be generated through hysteresis losses or frictional heating, referred to as dielectric hysteresis heating in non-conductors, as the susceptor moves against physical resistance in the surrounding material. Consideration of Joule heating alone results in a formula for the power-density P (W/cm³) in the inductively-heated material:

P=4πH² μo μr f M,

where H is the root-mean-square magnetic field intensity (A/m), f is frequency (Hz), M is a power density transmission factor (unitless) which depends on the physical shape of the heated material and skin depth and diameter of the part to be heated (4-5).

M, which is equal to the product of F and d where F is a transmission factor and d is the diameter of the part, can be shown to be maximally about 0.2 when the object diameter is 3.5 times the skin depth and when certain other assumptions are made.

Thus, for a given frequency there is a diameter for which the power density is a maximum; or equivalent, there is a maximum frequency for heating a part of a certain diameter below which heating efficiency drops dramatically and above which little or no improvement of heating efficiency occurs. It can also be shown that the power density of inductively heated spheres is much higher than solid spheres of the same material.

Conventional applications of induction heating involve welding, hardening, brazing or forging metal components. Some applications have been reported which use the process to cure adhesives in bonding processes or for applying coatings. U.S. Pat. No. 6,348,679 discloses compositions used in bonding two or more conventional materials where the interposed composition consists of a carrier and a susceptor, which may be at least in part composed of certain proteins. However the applications apply to conventional substrates such as films, metal substrates or wood.

There are only a few examples of the use of inductive heating in medical literature or for applications with biological materials. Principles of inductive heating have been applied to hyperthermia of cancer whereby large metallic “seeds” are inductively heated using a coil external to the body (6-7). Additionally, a recent report described the use of induction heating to heat nanocrystals coupled to DNA to locally denature DNA for the purpose of hydridization (8).

U.S. patent application Ser. No. 2002/0183829 describes inductively heating stents made of alloys with a high magnetic permeability and low Curie temperature for the purpose of destroying smooth muscle cells in restenosing blood vessels.

Common problems exist throughout the prior art. These include tissue damage due to uneven heating, unknown and/or uncontrollable thermal history, i.e., time-temperature profile, and relatively high cost. It is notable that a consistent means of treatment and control are desirable. The Code of Federal Regulations, 21 CFR 860.7(e)(1), establishes that there is “reasonable assurance that a device is effective when it can be determined, based upon valid scientific evidence, that in a significant portion of the target population, the use of the device will provide clinically significant results.” Devices that cannot be shown to provide consistent results between patients, or even within a patient upon multiple use, will have minimal utility and may not be approved, if approved, for broad use. Beyond devices, it is generally desirable to develop medical products with critical controls that can deliver precise results.

A tissue fusion wound closure device that overcomes the many deficiencies described in the prior art would improve patient care and reduce costs while supporting the expanded use of minimally invasive surgery. The inventors have recognized an increased need for a closure device and method that maintains the clinical advantages of laser-tissue welding, but eliminates the limitations. The prior art is deficient in devices and methods for minimally-invasive methods that use electromagnetic energy to controllably alter a biocompatible structure through molecular alterations and/or mechanical shrinkage to adhere to tissue. The present invention fulfills this longstanding need and desire in the art.

### SUMMARY OF THE INVENTION

The present invention is directed to a device to effect fusion between a tissue and at least one substrate to treat the tissue in an individual. The device comprises a fusion composition, a means to deliver a high frequency voltage or current to effect fusion and a means to control the extent of fusion. The device further may comprise a conductive material embedded within or proximate to the fusion composition.

The present invention also is directed to a device to effect a weld between a tissue and a substrate to treat the tissue in an individual. The device comprises a fusion composition or a conductive material or a combination thereof, a means to deliver a high frequency voltage or current to effect fusion and a means to control the extent of fusion.

The present invention is directed further to a device to heat biological materials comprising a fusion composition or a conductive material or a combination thereof and a means to inductively heat the fusion composition.

The present invention is directed further directed to methods of treating tissue or heating biological materials using the devices described herein.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1A depicts the placement of exposed terminals attached to an electrical conducting element within a material which is flowable upon the application of electromagnetic energy.

FIG. 1B is a cross-sectional schematic of a patch that is placed on the skin of an individual; the patch contains the electrical conducting element and a semi-permeable material.

FIG. 2 depicts the electrical conducting element with a linear geometry (FIG. 2A), with a coiled geometry (FIG. 2B) or consisting of small three-dimensional conducting nodes connected by fine linear elements (FIG. 2C).

FIG. 3A depicts a particular geometry of the electrical conducting element within a patch that is conducive to non-uniform heating.

FIG. 3B illustrates the theoretical temperature profile across the cross-section A-A of the patch in FIG. 3A.

FIG. 4A shows the conducting element positioned within a fusion composition in close proximity to the surface of the skin.

FIG. 4B shows the conducting element within a fusion composition in a coiled configuration to efficiently inductively absorb ambient radiofrequency energy produced by a coil attached to a radiofrequency power-source.

FIG. 4C depicts the conducting element within a fusion composition connected to a battery that is also incorporated into the patch.

FIG. 5 depicts a cross-sectional view of the patch showing that the fusion composition contains small conducting absorbers and an inductive coil around the fusion composition; the coil is powered by a battery regulated by an external switch.

FIG. 6 depicts a patch with an annulus for the weld connected to the terminals where a material or a medicament is contained within the annulus.

FIG. 7A depicts an arbitrarily shaped fusion composition containing an array of fine conducting elements. FIG. 7B depicts the placement of the array-containing fusion composition within the patch; a second part of the patch placed over the fusion composition contains conducting elements to heat the solder conductively or inductively.

FIG. 8 depicts the fusion composition containing an array of microneedles to alter skin surface prior to welding the fusion composition and the tissue. The fusion composition is surrounded by an annular electrode which incorporates an electrically conductive fluid.

FIG. 9A depicts the positioning of an active electrode within the fusion composition and the ground electrode emplaced on the stratum corneum distal to the fusion composition.

FIG. 9B depicts the positioning of both the active and ground electrodes within the fusion composition of FIG. 9A.

FIG. 10 illustrates the thermal history or temperature as a function of time of the fusion composition and contacting tissue. T1 is the ambient temperature of the fusion composition and contacting tissue, T2 is the threshold temperature T2 for the beneficial chemical change and T3 is the temperature at which irreversible thermal damage to extraneous tissue occurs. The duration of heating cycles illustrated may range from microseconds to milliseconds.

FIG. 11 depicts a solenoid-type coil applicator carrying an electrical current and the resultant magnetic field lines.

FIG. 12 depicts a coil applicator that can be split thus allowing positioning of tissue in the interior of the coil.

FIGS. 13A-13C depict configurations of three flat pancake coils.

FIGS. 14A-14C depict a pancake coil with a non-planar geometry (FIG. 14A), a conical spiral coil geometry (FIG. 14B) and a coil suitable for use within tubular structures such as blood vessels (FIG. 14C).

FIG. 15 depicts an ovine blood vessel anastomosed with an activator, applicator and fusion composition.

FIG. 16 depicts a histologic section through a blood vessel anastomosed with the invention.

### DETAILED DESCRIPTION OF THE INVENTION

One embodiment of the present invention provides a device for treating tissue in an individual to effect fusion between said tissue and at least one substrate comprising a fusion composition; a means to deliver a high frequency voltage or current to effect fusion; and a means to control the extent of fusion. The substrate may be a tissue, a dressing or a fastener. The device may be in a patch.

In all aspects of this embodiment the fusion composition and the conductive element independently may be at least one of a protein, a ferromagnetic material, a pharmaceutical, a conducting polymer, or an ionic solution. Representative examples of a protein include collagen, fibrin, elastin and albumin. Examples of a conducting polymer are hydrogel, sol-gel or a synthetic biomolecule. The conductive material may be a metal, a protein, a ferromagnetic material, a pharmaceutical, a conducting polymer, or an ionic solution. Additionally, the conductive material may be embedded within the fusion composition or may be separate from but proximal to the fusion composition.

Further in this embodiment the means to deliver a high frequency voltage or current may be at least one active terminal, a battery or an active electrode and a ground electrode. Alternatively, the active terminal may be an electrode array having a plurality of isolated electrode terminals. In an aspect of this embodiment both the active and ground electrodes are embedded within the fusion composition. In another aspect of this embodiment the active electrode is embedded within the fusion composition and the ground electrode is located distal to and external to the fusion composition. Still within this embodiment the means to control the welding process may be electronic, a means to monitor the thermal history of the device or a means to detect changes in a ferromagnetic material which comprises the fusion composition, the conductve material or both as the Curie temperature of the ferromagnetic material is reached.

Another embodiment of the present invention provides a method of treating tissue in an individual by effecting a weld between the tissue and at least one substrate, comprising the steps of placing the device described supra on the tissue of the individual; delivering the high frequency voltage or current to the fusion composition comprising the device; and monitoring the device to control the extent of the weld between the tissue and said substrate(s). The steps of the delivering the voltage or current and monitoring the device may be repeated at least once.

Yet another embodiment of the present invention provides a device to effect a weld between a tissue and a substrate to treat the tissue comprising a fusion composition or a conductive material or a combination thereof; a means to inductively generate heat to effect the weld, and a means to control the extent of the weld. In all aspects of this embodiment, the substrates, the fusion composition, the conductive material and the location of said are as described supra.

In this embodiment the means to inductively generate heat comprises an induction coil to receive radiofrequency energy which is proximate to the device. The induction coil further may comprise a clamp-like instrument having two arms pivotally connected at the center. The first ends of the arms are attached to the induction coil and the second ends of the arms are utilized to manipulate and position the inductive coil proximate to the fusion composition and/or the conductive material. The induction coils may be coated in a smooth non-adhering material. Examples of a non-adhering material are teflon, titanium, glass, cadmium, chromium, polyethylene glycol, alginate or gold.

The application of the radiofrequency energy may be controlled by circuitry such as a battery and switch. Additionally, the induction means may also have a feedback control circuit to monitor voltage and conductance. The means to control the extent of the weld in this embodiment is as described supra.

Yet another embodiment of the present invention provides a method of treating tissue in an individual to effect a weld between a tissue and a substrate, comprising the steps of placing the device disclosed supra on the tissue of said individual; inductively heating the fusion composition and/or the conductive material comprising the device; and monitoring the device to control the extent of the weld between said tissue and said substrate(s). The steps of inductively heating the fusion composition and/or the conductive material and monitoring the weld process may be repeated at least once.

Still another embodiment of the present invention provides a device to heat biological materials comprising a fusion composition or a conductive material or a combination thereof and a means to inductively generate heat to effect heating of the biological materials. The device may further comprise a means to control the extent of heating. Examples of such means is electronic, a means to monitor the thermal history of the device or a means to detect changes in a ferromagnetic material comprising said fusion composition. The biological material may be a tissue, a dressing or a fastener. The fusion composition, the conductive material and the position thereof with respect to the fusion composition, the induction coil and the coating and components thereof are as described supra.

Still another embodiment of the present invention provides a method of heating biological materials comprising the steps of placing the device described supra proximate to the biological materials; and inductively heating said fusion composition or said conductive material comprising the device or a combination thereof to effect heating of the biological materials where the step optionally may be repeated at least once. This embodiment further may comprise the step of monitoring the device to control the extent of heating where the step optionally may be repeated at least once.

As used herein, the term “weld” or “solder” may be used interchangeably to represent bonding, fusing or attaching of one or more substrates including sections of tissue to another section of tissue, to a dressing, or to a fastening device such as a clip, pin or staple.

The present invention generally relates to a device and method for heating a liquid, solid or semi-solid fusion composition to be utilized as a means of heating biomolecules, particularly those in living systems. The device may consist of a source of electrical energy coupled to at least one electrode or a source of radiofrequency (RF) energy coupled to an applicator or induction coil to generate an electromagnetic field. Electrical energy or the oscillating magnetic field interacts with the fusion composition resulting in the production of heat substantially within the fusion composition.

The consequence of heat is molecular changes in the composition resulting in fusion with the adjacent tissue. The adjacent tissue may take part in the fusion process by also being altered by the transient presence of heat. The heating process can be used to heat tissue components, such as proteins, lipids and carbohydrates, such that they may be altered in structure, adhere to one another, or be separated from one another. Applications include, but are not limited to, bonding, coagulating, filling in tissue defects, anastomosis, and separating tissue components.

Provided herein are devices and methods for heating non-conventional substrates, i.e. biological materials, in order to cause conformational changes that result in unique properties with regard to tissues. The device, in addition to the fusion composition, may comprise components, such as electrodes, to conductively heat the biological materials or, preferably, may comprise an applicator to inductively heat the biological materials to cause them to join to one another or to non-biological materials. Further, the device requires a power source or activator through which to deliver the electric current to the electrodes or to generate radiofrequency energy to induce an oscillating magnetic field.

As such, the present invention provides devices and methods for joining and fusing biological tissues to each other by heating the tissues in the presence of a fusion composition or material that promotes the formation of a strong weld. The fusion composition may be placed between layers of tissue or between a tissue and a dressing that are to be welded or fused. For wound closure a dressing or other fastener containing such fusion composition may be applied to the wound site and welded in place.

The materials that comprise the fusion composition must be biocompatible, able to be inductively heated and able to produce a fusion in biomaterials. The fusion composition may comprise a biocompatible polymer, a protein such as albumin, elastin and/or collagen or polysaccharides, e.g. cellulose, starch, chitosan, alginate, emulsan, or pectin. Examples of biodegradable polymers are polylactide (PLA), polyglycolide (PGA), lactide-glycolide copolymers (PLG), polycaprolactone, lactide-caprolactone copolymers, polyhydroxybutyrate, polyalkylcyanoacrylates, polyanhydrides, and polyorthoesters. Examples of biocompatible polymers are acrylate polymers and copolymers such as methyl methacrylate, methacrylic acid, hydroxyalkyl acrylates and methacrylates, ethylene glycol dimethacrylate, acrylamide, bisacrylamide or cellulose-based polymers, ethylene glycol polymers and copolymers, oxyethylene and oxypropylene polymers, poly(vinyl alcohol), polyvinylacetate, polyvinylpyrrolidone and polyvinylpyridine. Optionally, protein primers, which are substances that exhibit groups that can cross-link upon the application of heat, can be added.

Proteins are particularly attractive in tissue bonding applications in that they typically denature at temperatures less than 100° C. Denaturation can lead to cross-linking with other molecules, particularly proteins, in the immediate environment while the proteins are still in the denatured state, or upon their renaturation. Additional materials added to the composition formulations may result in greater flexibility and tensile strength as well as optimum treatment times and temperatures. The fusion composition optionally may be charged, for example, when not at its isoelectric point, or may have charged molecular species present which interact with the electromagnetic field.

The formulations utilize commonly occurring tissue and proteins, such as albumin, collagen, elastin, but may also contain silk, lignin, dextran, or may contain soy-derivatives, polyglutamic acid, combined with additives such as polyethylene glycol or hydrogel to improve the rheologic nature of the adhesive. The biocompatible proteins preferably are elastin, albumin or collagen and are present at concentrations of about 1% to about 75% and more preferably 50-75%.

Optionally, hyaluronic acid can be added to the composition to enhance the mechanical strength of adhesives, such as is sometimes done in laser tissue welding, or pre-denaturation may take place before application of the composition at the treatment site. Other materials, such as fibrinogen or chitin or chitosan, may be added to the composition to provide hemostasis and/or some degree of immediate adhesion. Materials such as calcium phosphate or polymethylmethacrylate, also can be used, most beneficially when boney material is the tissue to be treated.

Additionally, pharmaceuticals, e.g., an anti-coagulant, an antithrombotic, an antibiotic, a hormone, a steroidal anti-inflammatory agent, a non-steroidal anti-inflammatory agent, an anti-viral agent or an anti-fungal agent, may be beneficially added to the composition in order to provide some desirable pharmacologic event.

Optionally, destabilizing/stabilizing agents, e.g. alcohol, can be added as they have been shown to alter the denaturation temperature. For example, an increase in the concentraion of NaCl, referred to as “salting-in” proteins, can increase the denaturation temperature of lactoglobulin, while an increase in the concentraion of NaClO4, or “salting-out”, reduces the denaturation temperature (9). When proteins are exposed to either liquid-air or liquid-liquid interfaces, denaturation can occur because the protein comes into contact with a hydrophobic environment. If allowed to remain at this interface for a period of time, proteins tend to unfold and to position hydrophobic groups in the hydrophobic layer while maintaining as much charge as possible in the aqueous layer. Thus, by ultrasonically adding bubbles to the composition will serve to lower the denaturation point of the mixture.

The fusion compound may further comprise an electrically conductive element. The conductive materials that can be inductively or conductively heated are added to the fusion composition in amounts typically in concentrations of from 0.1 to 25%. Higher concentrations may be used under circumstances where effects of the conductive materials on living systems are not a factor. The material may be composed of salts or other ionic substances, or metals of variable size, depending on the operational frequencies. Additionally, the metallic materials may be an alloy with a Curie point in the range of about 42° C.-99° C. Generally, the range of useful particle sizes are from nanometer size to macroscopic size particles up to 1 mm wide. The particles may be, but not limited to, spheroidal, elongate or flakes. Alternatively, the conductive material may take of the form of a fine mesh or film, such as available from Alfa Aesar Inc (Ward Hill, Mass.).

Example of materials that may be useful by themselves, or in alloys, in the present method and composition are tantalum, niobium, zirconium, titanium, platinum, Phynox (an alloy of cobalt, chromium, iron, nickel, molybdenum), palladium/cobalt alloy, magnetite, nitinol, nitinol-titanium alloy, titanium (optionally alloyed with aluminum and vanadium at 6% Al and 4% V), tantalum, zirconium, aluminum oxide, nitonol (shape memory alloy), cobalt (optionally alloyed with chromium, molybdenum and nickel, or optionally 96%Co/28% Cr/6%Mo alloy), iron, nickel, gold, palladium, and stainless steel (optionally biocompatible type 316L). The conductive materials may take the shape of a mesh, fibers, macroscopic and solid materials, flakes or powder. The conductive materials may be anodized and may further be encapsulated in materials such as liposomes, compounds such as calcium phosphate, polystyrene microspheres, pharmaceuticals, hydrogels, or teflon. The conductive materials may also be complexed with glass and ceramics. These complexes and encapsulating materials may minimize immune responses, or toxic reactions to the conductor, could induct a desirable pharmacologic event, or could enhance the inductive coupling to the activating magnetic field.

The rheology of the fusion composition can be important. For example, producing the composition in a low-viscosity liquid form would allow injection through a cylindrical pathway such as a trocar or working-channel of an endoscope. A higher viscosity material can be applied to a tissue and will stay in place prior to activation. A solid formulation could be shaped, for example, as a tube, which could be positioned in a tubular anatomical structure, e.g. a blood vessel or ureter, thus providing mechanical support prior to activation.

Other shapes may be more appropriate for different procedures. For example, a flat-sheet of composition would be suitable for sealing a large area of skin or soft-tissue, while a solid cylinder could be most appropriate for placement in the cavity left behind after a cannula is extracted. Alternately, the material may be molded into a tape which can be applied to conform to the surface of planar and irregular-shaped objects. A porous structure of the fusion formulation might be beneficial for the subsequent in growth of cells. It is contemplated that the conductive material itself, when distributed throughout the treatment area, would utilize the endogenous proteins in production adhesion thus precluding the use of an external protein in the formulation.

Optionally, the composition may have different additives depending on the material to which adhesion is required. For example, vascular graft materials composed of polytetrafluoethylene (PTFE) or Dacron may complex with denatured albumin. Alternately, gelatinized PTFE, when used as one of the components of the fusion composition, could adhere to the PTFE in situ, thus effecting the desired result. Heat-curable adhesives also may be included in the fusion composition. For example, heat-curable polymethylmethacrylate (PMMA) may be used to fuse bone components to one another or to fill defects.

The fusion composition may incorporate a support lattice, such as can be made from, for example, polylactides, silk, PTFE or dacron, or a conductive material such as fine stainless steel mesh. The support material would allow for the fusion composition to be formed into a particular shape suitable for application to a particular anatomical structure. A conductive lattice would allow for inductive heating as well as mechanical support. Additionally, the efficiency of heating the fusion composition may be improved through the addition of ions in sufficient concentration to result in dielectric heating whereby ionic conductivity serves as a “bridge” between small particle conductive materials in the fusion composition.

The device may be in a patch to be used externally or a small patch to be used endoscopically. Although not limited to such a configuration a patch provides an excellent means to effect, inter alia, wound closure via conductive heating of the fusion composition, although inductive heating of the fusion composition is not precluded in a patch. However, many different arrangements of the elements within the patch are possible and each arrangement would have a particular feature beneficial in certain circumstances. An electrically conductive element or material terminating in exposed terminals may be incorporated into a material. The conductive element may be coupled to a current source or high frequency voltage source through the terminals. The conductive element may be linear, coiled, or consist of small three-dimensional conducting nodes connected by fine linear elements. Alternatively, the conducting element is arranged within the patch in a particular geometry to result in a non-uniform heat and, thus, weld across the area of the patch.

Upon being exposed to electromagnetic energy, or to the heat generated therefrom, the molecules in the material containing the electrically conductive element change in conformaton, altering their interaction with each other or with molecules in the surrounding environment. For example, upon heating, protein may become more fluid, and flow into a second material, whereupon the molecules assume a different conformation upon cooling, thus enabling them to cross-link with molecules in the second material. The second material may be composed of tissue, or may comprise, for example, a semi-permeable structure of carbon, of ceramic or of a polymer lattice such as a sol-gel or hydrogel. Additionally this second material may be an electrically conducting fluid or medicament that provides a pathway for electrical energy to reach the skin and effect tissue alteration, e.g., denaturation, thereby effecting a tissue-weld.

Alternatively, the electrical energy applied to the conductive element is provided by a battery incorporated into the patch. Given that the temperature rise necessary to cause the beneficial thermal alterations in the fusion composition are no more than about 60° C., and more likely only about 30° C., the energy available in the battery can be low enough that only a very small battery is required. This results in a convenient to use and yet disposable patch.

The tissue fusion composition may be heated by alternate means. The device effects thermal changes in the fusion composition, which is placed between the tissues, e.g. skin, or dressing to be welded, through inductive heating of small, conducting absorbers within the fusion composition or, alternatively, of the fusion composition which is the conducting element itself. Representative examples of the tissue fusion compositon are collagen, fibrin, elastin, and albumin. Medicaments may also be incorporated within the fusion composition. The conducting absorbers or conductive material within the fusion composition may be, for example, ferromagnetic materials such as iron or copper, or biocompatible ionic species such as sodium chloride or biocompatible nonionic compounds with high dipole moments.

The conducting element may also have a geometry, e.g. a coiled configuration, that efficiently inductively absorbs ambient radiofrequency energy. For example, a coil which is attached to a radiofrequency power-source external to and superimposed proximally to the patch will produce a magnetic field around the fusion composition. The conductive element is thus heated leading to thermal alterations of the tissue fusion composition material which then effects a tissue-weld at the surface of the skin. The conductive element may also provide a means of measuring the heat generated in the system allowing for monitoring at a distal location. The conducting element may optionally be removed after the tissue fixation treatment, through physically withdrawing the element or through dissolving and absorption as a result of physiological processes. This may be accomplished, for example, through the use of conductive metals and polymers that are either solid or mixed in a semi-solid matrix.

Heating also may be effected by applying radiofrequency energy to a coil positioned around the fusion composition thus causing a strong and alternating magnetic field within the fusion composition. This radiofrequency energy can be produced through circuitry powered by a battery and modulated with an external switch. For example, using a ferromagnetic material within the fusion composition, the fusion composition is heated by the external magnetic field until it reaches the Curie temperature of the ferromagnetic material at which point the heating ceases until the material cools below it's Curie temperature whereupon the heating cycle can be repeated.

It is additionally contemplated that the weld that holds the patch in place may take the form of an annulus. Positioned within the annulus is a material or medicament that is beneficial to wound healing. Examples of this material or medicament are a hydrogel or antibiotic ointment. Alternatively, the fusion composition may have an arbitrary shape and may or may not contain a medicament. The fusion composition incorporates an array of fine conductive elements such as, for example, metal or magnetic particles that may be heated by induction, or a series of metal wires or mesh that may be heated conductively. The fusion composition can be cut with a scissors and placed over the wound to be treated. A second part of the patch is placed over the fusion composition and is used to inductively or is used to conductively heat the fusion composition through the application of radiofrequency energy or electrical energy via the terminals in the patch thereby effecting the tissue weld.

In other aspects of the invention, the fusion composition is optional, or may be composed simply of a conductive material. For example, tissue fusion may be accomplished by applying metal particles to the interface between two tissue faces, or between tissue and another material, and, upon application of an alternating magnetic field, e.g. induction, the heat generated in the metal will diffuse to the surrounding tissues to create a weld.

The RF device used in these embodiments may provide for a continuously delivered magnetic field, such as is delivered through conventional induction heating and RF surgical devices. Alternatively, a pulsed field may be provided as, for example, is generated by diathermy devices. Pulsed fields may alternatively be generated using capacitors in a cyclic manner to successively charge and release current to the respective RF generating devices. In this manner, large currents may be generated over brief amounts of time, with successive pulses. Pulsing the device in this manner also serves to minimize the effects of heat diffusion, over relatively long periods of time, to surrounding tissue, by minimizing the duration of exposure to heating.

In order to effect a strong weld, it may be beneficial to pre-treat the skin surface before altering the tissue fusion composition and tissue whereby the weld takes place. The patch may contain an array of microneedles within a tissue fusion composition surrounded by an annular electrode which incorporates electrically conductive fluid. Upon the application of radiofrequency energy or a brief, e.g., a few microseconds, pulse or bipolar pulse of direct-current, tissue alterations take place in the skin concomitant with thermal changes to the fusion composition.

Additionally, electrodes can be excited by radiofrequency energy or a pulse or bipolar pulse of direct-current, whereupon a plasma is formed between the active and ground electrode. This creates alteration to the stratum corneum as well as beneficial changes to the fusion composition while leaving the epidermis unharmed. The plasma may also lead to the formation of transient cavitation bubbles that can also induce beneficial changes in the stratum corneum and/or fusion composition.

The device may also comprise a heating element with impedance greater than tissue. The heating element is electrically positioned in series with a tissue, a conductive element and a second conductive element of lower resistance so that current flows through the tissue and the first element resulting in preferential heating of the element. A second conductive element with impedance less than tissue is in electrical series and grounds the current. Alternatively, a heating element with an impedance less than tissue is positioned electrically parallel with a tissue. Current flows through the tissue and heating element preferentially heating the element; a further conductive element with an impedance less than the tissue and the heating element taken together is in electrical series and grounds the current.

A safety interlock may be integrated into the patch such that the device cannot be utilized unless the interlock is engaged, and only under proper use. For example, the interlock could be mechanical, electrical or optical. In the “on” position (engaged or disengaged), the device may be operational. In the “off” position, the device would fail to be operational. This could prevent unauthorized use and would prevent the device from being used twice which would be unsanitary.

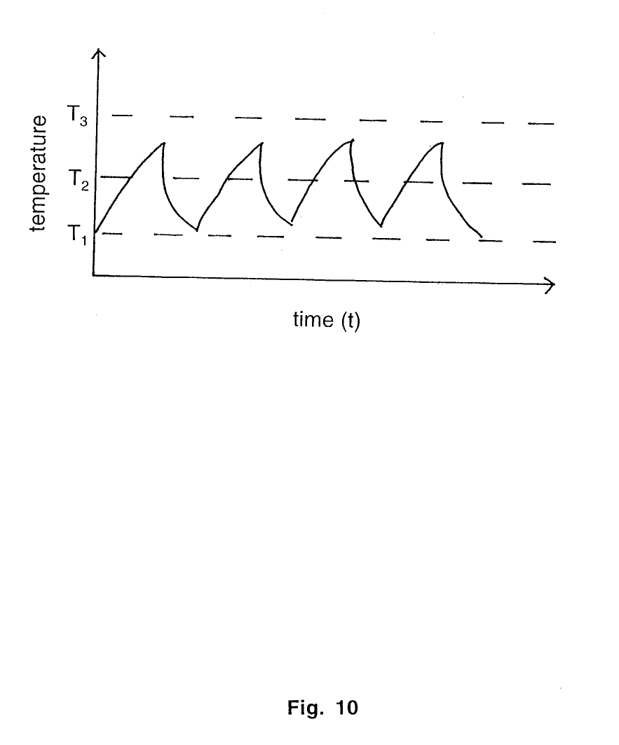

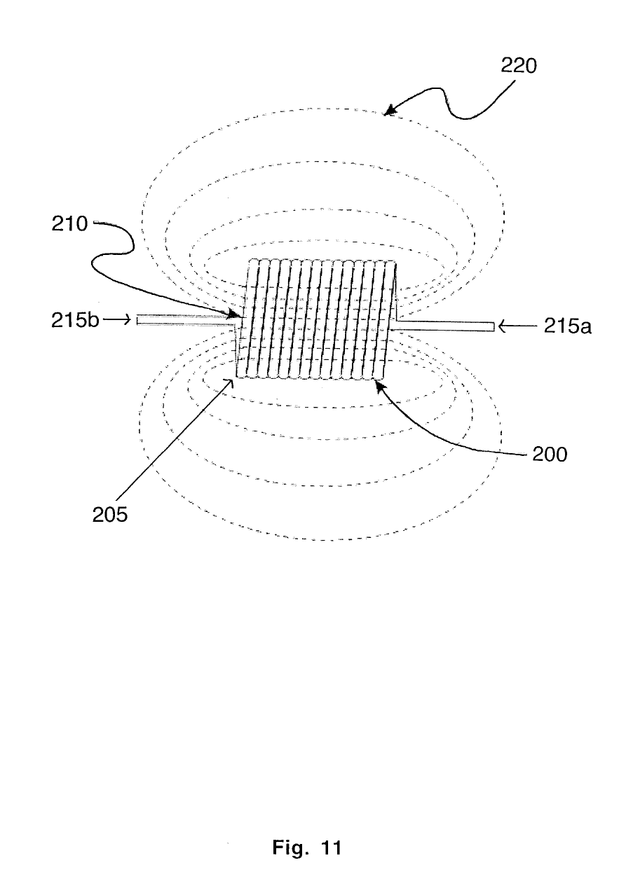

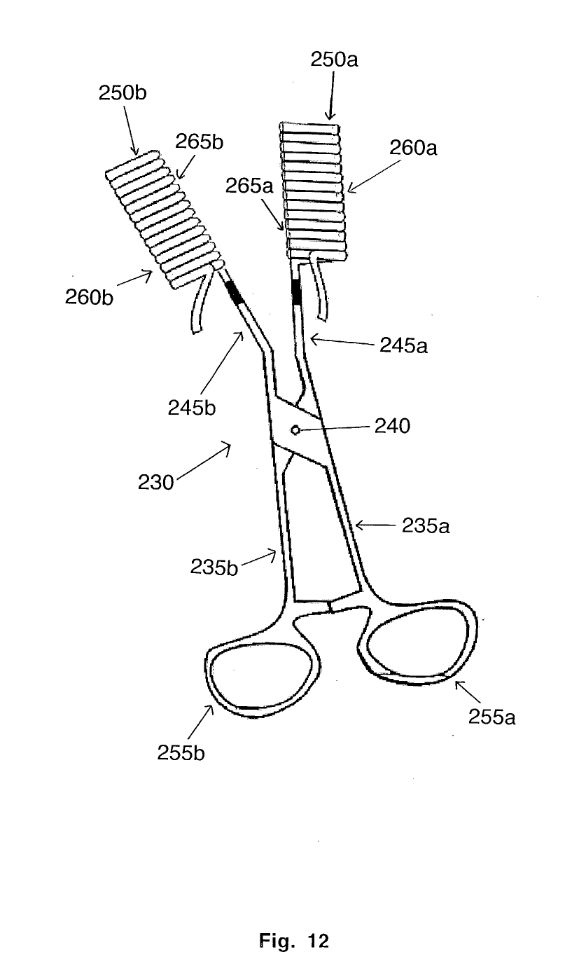

The present invention also provides a means to control the welding process by monitoring and regulating the heat generated or used in the system, so as to avoid overheating and damage to the substrates, and to provide a uniform weld. The thermal history, i.e, temperature as a function of time, of the fusion composition and contacting tissue must be such that the beneficial chemical changes take place, e.g., denaturation, and yet little or no extraneous heat is produced which could otherwise lead to unwanted extraneous thermal damage (FIG. 10). According to Arrhenius Rate Theory, the rate of a chemical reaction is exquisitely sensitive to temperature, but only linearly related to the time that a particular temperature is held. Thus, it is of benefit to quickly heat the tissue and tissue fusion composition from their ambient temperature T1 to a temperature beyond the threshold temperature T2 for the beneficial chemical change, but not beyond the temperature T3 for irreversible thermal damage to extraneous tissue.

Once the critical temperature T2 is exceeded, the device quickly cools because of the small mass of the conductive heating elements or absorbers within the fusion composition whereupon the heating cycle can repeat. When the heating is done in a time more rapid than the time it takes the heat to conductively dissipate out of the heated tissue and fusion composition, then the total amount of energy used and heat produced during the process is minimized. Depending on the thermal properties of the heating elements and tissue, the duration of these heating cycles may be as short as microseconds or as long as milliseconds and the heating cycle can be repeated as many times as required to effect a suitable tissue fixation.

The tissue welding process also can be monitored by changes in the electrical properties of the electromagnetic circuit that is made up of the power supply, induction coil, material to be heated by the coil and the body. These changes may include but not be limited to changes in voltage or conductance or changes in the magnetic properties of a ferromagnetic material in a fusion composition as it reaches its Curie temperature.

The power supply used may be a constant current or a constant voltage power supply or may be a modulated current or a modulated voltage power supply. Also the conductive or inductive heating process can be monitored by sampling changes in the first and/or second time derivative of the impedance of the tissue, comparing this derivative to zero and using this information to modulate the heating process.

Preferably, the instant invention provides a device comprising a source of radiofrequency (RF) energy coupled to an applicator, which then produces an oscillating magnetic field, and the fusion composition which inductively couples with the magnetic field, resulting in the transient production of heat substantially within the composition. Inductive coupling most simply results in heating through the magnetization of particles or other ionic species, either with non-zero conductivity and magnetic permeability, impregnated in a biocompatible fusion composition or adhesive. Thus, a basic tissue fusion device (TFD) to inductively fuse or bond biological materials comprises the fusion composition, an applicator and an activator. The device may create a weld or a bond between tissues or between tissue and some other material.

The fusion composition may be composed largely of a protein, such as serum albumin, with the addition of a metal such as 300 mesh nickel flakes. The induced electrical currents produced in the particles results in heat which then conducts into the area immediately surrounding the metal, resulting in a “melting” of the adhesive and perhaps the adjacent tissue. When the adhesive cools, less than a second later, it forms a bond with the tissue, perhaps through cross-linking of the proteins. In tissue, we hypothesize that the temperatures needed to achieve a bond range from about 45-85° C., and the heating times are very short since protein denaturation is essentially instantaneous once a critical temperature is achieved. Thus, the powers required for the present device and method are far less than those used in commercially available industrial induction-heating devices which are used for welding metals and plastics. Accordingly, the present invention can be produced for a fraction of the cost of commercial devices.

Applicator geometry greatly affects the distribution of the resultant electromagnetic field. There are several different possible designs for the applicator. The most efficacious design depends on the procedure for which it is intended to be used. In the case of induction heating, a coil of wire can be connected to the activator in order to produce a strong and uniform magnetic field along the long-axis of the coil and is most suitable for inductively heating materials positioned within the turns of the coil. Alternatively, the magnetic field can be externalized from the interior of the coil with the use of a core material, such as used in transformers. The core material may be of a magnetic material, and optionally a powdered magnetic material, so that heat production in the core is minimized.

The source of RF energy may provide electrical energy to a probe that may be an electrically conducting material, such as copper, wound in the shape of a solenoid or coil. Other probe shapes may be more suitable for particular applications. The conducting material sets up an oscillating magnetic field which inductively couples to a conductive material in the composition. Heat is produced through physical movement of the conducting material and/or the establishment of Eddy currents within the conducting material or the tissue and/or fusion composition and/or hysteresis losses. The heat diffuses into the surrounding composition and tissue thereby causing protein denaturation and subsequent molecular bonding thus effecting adhesion.

The conducting material comprising the probe may be hollow and so a cooling fluid can be circulated within its lumen or the probe may be solid. Alternatively, cooling is enhanced by using a hollow tubing, such as copper, through which a cooling fluid such as water can be circulated. If required, the coil can be cooled by encapsulating it in a glass envelope through which a cooling fluid with a low electrical permitivitty, such as low viscosity mineral oil, can be circulated.

Alternatively, the coil can be made in such a way that it can be opened up thus allowing a tissue, such as a blood vessel, to be positioned within the coil which then closes and completes the circuit. Other applicator designs allow for a relatively strong magnetic field to be produced exterior to the wire or tubing. For example, the designs of applicators may be such that the field is produced above or below the plane of the conductor. In a coil with a butterfly configuration, the strongest field is produced below each separate coil while in coils with spiral configurations, the strongest field is produced in a single position below the coil.

Optionally, the applicator can be bent into a particular shape whereupon the distance between the material to be heated and the conductor that makes up the applicator is minimized. This provides for an efficient use of energy. Also, optionally, a ferromagnetic material, e.g. pole-piece, may be partially positioned in the magnetic field produced by the applicator, thereby allowing the field to be transferred to the end of the pole-piece thus producing concentration of the field lines and providing greater accessibility to the field. At high frequencies, it may be beneficial for this pole piece to be made substantially from powdered ferromagnetic materials in order to minimize undesirable heating in the pole piece itself.

An oscillating magnetic field may be applied using an instrument having two separate coils attached independently to the ends of a clamp-like extension or, alternatively, a single coil may be made in such a way that it can be opened up thus allowing a tissue, such as a blood vessel, to be positioned within the coil which then closes and completes the circuit. The coils may be coated in a smooth non-adhering material which comprises, for example, teflon, titanium or gold. Using the scissors-like action of the clamp, the instrument is positioned around and proximal to the biocompatible fusion material. The coils can be attached to a radiofrequency power supply or activator that produces the oscillating magnetic field within the coils. It is contemplated that such a device may be used to anastomose tubular structures such as blood vessels or ureters.

The power-supply is able to produce radiofrequency energy in the frequency range of of 100 kHz to 5.8 GHz, more preferably between 350-800 kHz, or at 869 MHz, 900 MHz, 2.4 GHz, 13.56 MHz or 5.8 GHz. The power in the range 1-5,000 W and may typically operate at frequencies of 100 kHz to 15 GHz. The power of the RF energy is in the range of 1-5000 W, and depending on the application, may be more preferably in the range of 100-500 W.

The best operating frequency depends, among other things, on the nature of the fusion composition to be heated, the geometry and chemical composition of the material to be heated, tissue to be fused, or the cavity to be filled. Regulatory issues also may be a factor in the choice of frequency. The output impedance of the power-supply is preferably matched to the input impedance of the applicator, described below. The power-supply has several safety features incorporated; for example, the output is optionally of low or moderate voltage, <240V, preferably no more than 50V, which is traditionally considered a safe voltage.

The device is shielded for emitted or received electromagnetic-interference. There are thermal switches incorporated within the device to shut it down if overheating occurs and there are fast breakers that quickly cut off the output if a power-output transient occurs. Multiple interlocks are incorporated in the device which prevent running the device with the cover removed. A footpedal is optionally incorporated in order to minimize the possibility of unintentional activation of the device.

Control may be exerted by direct feedback monitoring of heat generation or by prediction and measurement of the magnetization of the composition over time with regard to its volume and mass. This feedback may arise from measurements of impedance changes in the applicator, as the tissue becomes part of the circuit during treatment, or devices such as thermocouples or infrared thermometers may be utilized. A second order of control may be exerted through the use of ferromagnetic metals and alloys as susceptors which remain magnetized until reaching a critical temperature, the Curie temperature, whereupon the cease to be magnetic.

The fusion compositions and/or the conductive elements of the present invention may be used in methods of fusing, welding or creating a bond between tissues or between tissue(s) and another material such as, but not limited to, a tissue, a dressing, a fastener, or other biocompatible substrate. The fusion compositions can be used as a sealing agent to seal a sinus in a tissue, to aid in forming an anastomosis between tissues or as an adhesive to adhere a dressing or other wound covering or fastening material to tissue(s). Furthermore, the conductive material itself may function as a fusion composition. The conductive material, e.g., metal particles, may be placed on or between the tissues or tissue(s) and other substrates to inductively form a weld or seal or bond.

Additionally, the device may be used as a method of indirectly dissecting and/or cauterizing tissue, i.e., without directly contacting the tissue a cauterizing or dissecting instrument or agent. A conductive composition is applied to the surface of a substrate, such as a tissue which is leaking fluids, e.g., blood, whereupon the conductive composition is heated via induction to a point where the tissue beneath is cauterized as a result of the heat generated. Alternatively, the heat generated in the conductive composition may cause the tissue beneath to separate. Separation or dissection is followed by cauterization thereby limiting bleeding.

As described below, the invention provides a number of therapeutic advantages and uses, however such advantages and uses are not limited by such description. Embodiments of the present invention are better illustrated with reference to the Figures, however, such reference is not meant to limit the present invention in any fashion. The embodiments and variations described in detail herein are to be interpreted by the appended claims and equivalents thereof.

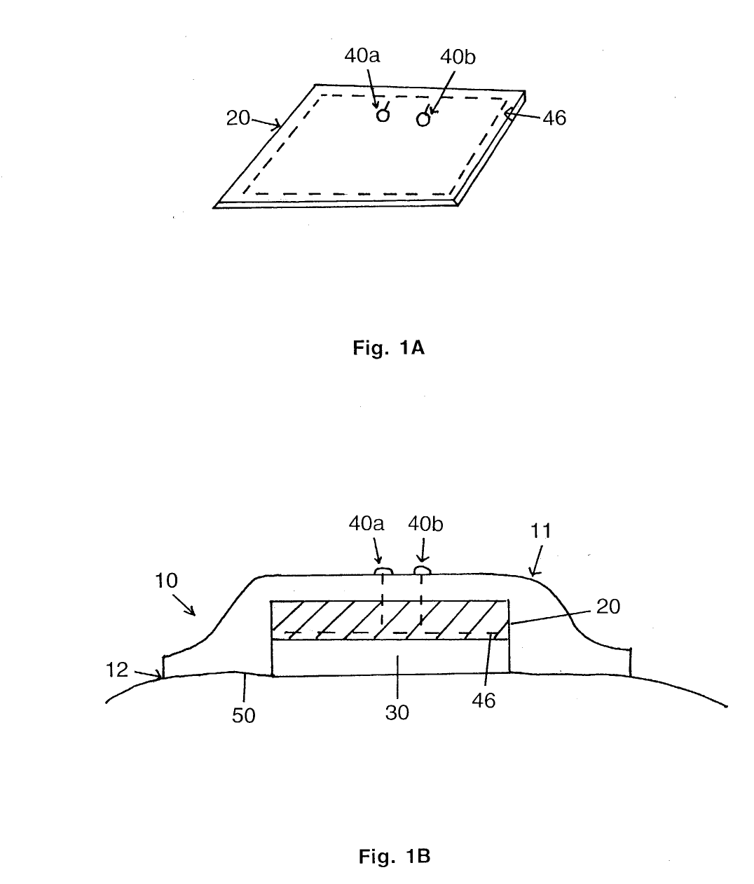

FIG. 1A shows a material 20 which may be a semi-solid matrix incorporating a conducting element 46. The conducting element terminates at exposed terminals 40a,b. The terminals 40a,b may couple the conducting element 46 to a current source or high frequency voltage source (not shown).

In FIG. 1B the material 20 containing the conducting element 46 is incorporated into a patch 10. The patch 10 has an upper surface 11 on which the terminals 40a,b are located and a lower surface 12 which contacts the surface of the skin 50. The patch may optionally have an adhesive for temporary adherence to the tissue. The material 20 containing the conducting element 46 is contained within the patch 10 and placed in contact with a fusion composition 30 within the patch 10 which is in contact with the skin 50 such that the fusion composition 30 is sandwiched between the material 20 and the skin 50.

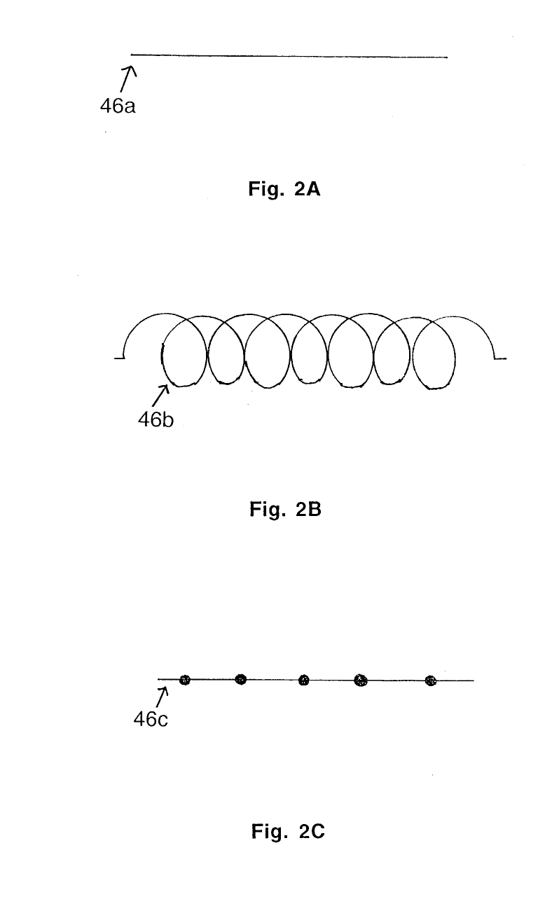

With reference to FIGS. 1A and 1B, FIGS. 2A, 2B and 2C depict possible geometries of the conducting element 46. The conducting element 46 may be linear 46a, coiled 46b or consist of small conducting nodes which are connected by fine linear elements 46c. It is to be noted that reference to conducting element 46 includes, but is not limited to, geometries 46a, 46b and 46c of the conducting element 46 unless specifically referenced otherwise.

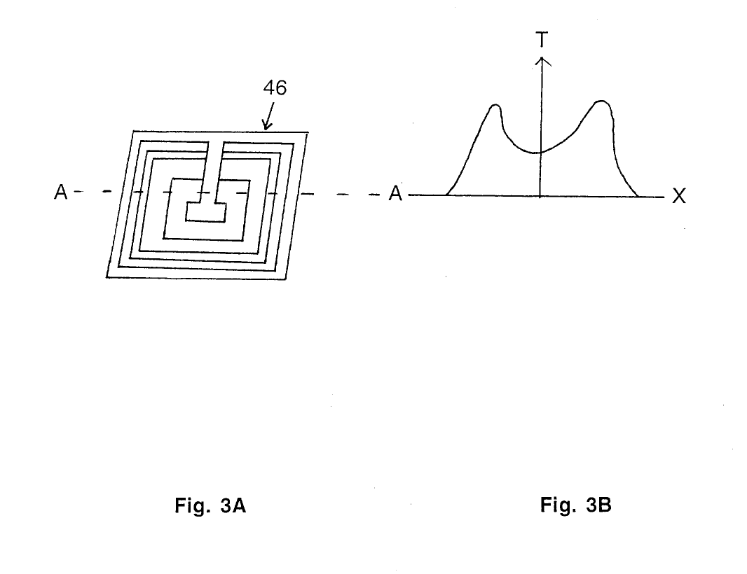

FIG. 3A depicts an arrangement of the conducting element 46 in the material 20 within the patch 10 (not shown) in a particular geometry that results in a non-uniform heating and, thereby, weld across the area of the patch 10. FIG. 3B illustrates a theoretical temperature profile across a cross-section A-A in material 20 of the patch 10 showing the non-uniformity of the temperature across the patch 10.

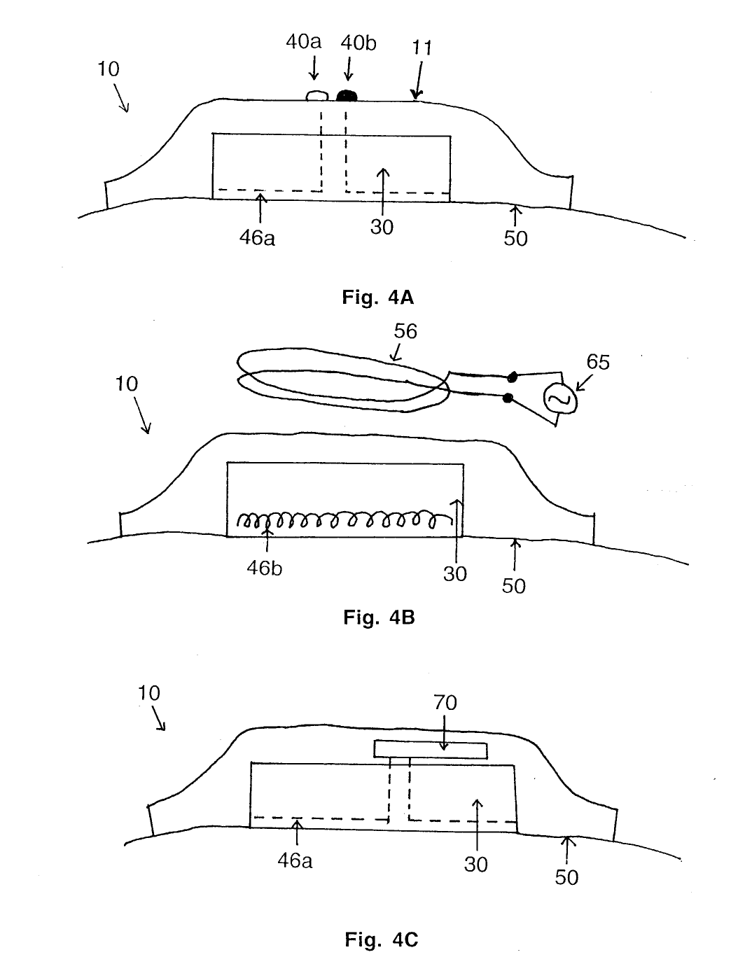

Still with reference to FIG. 1B, FIGS. 4A-4C depict a patch 10 having the conducting element 46 within the fusion composition 30 with various means of conductively or inductively heating the conducting element 46. In FIG. 4A a patch 10 comprises a fusion composition 30 placed within the patch 10 such that the patch 10 and the fusion composition 30 are in contact with the skin 50. The conducting element 46a is positioned within the fusion composition 30 to be in close proximity to the surface of the skin 50. The conducting element 46a terminates at exposed terminals 40a,b located on the outer surface 11 of the patch 10. The terminals 40a,b may be coupled to a current source or high frequency voltage source (not shown) as in FIG. 1B.

In FIG. 4B the fusion composition 30 contains conducting element 46b located proximally to the surface of the skin 50. The conducting element 46b inductively absorbs ambient radiofrequency energy generated by a coil 56. The coil 56 is external to the patch 10 and superimposed proximally to the upper surface 11 of the patch 10. The coil is attached to a radiofrequency power source 65.

FIG. 4C depicts a patch 10 with fusion composition 30 having a conducting element 46a as in FIG. 4A. The conducting element 46a terminates in a battery 70 incorporated into the patch 10 but external to and superimposed proximally to the fusion composition 30.

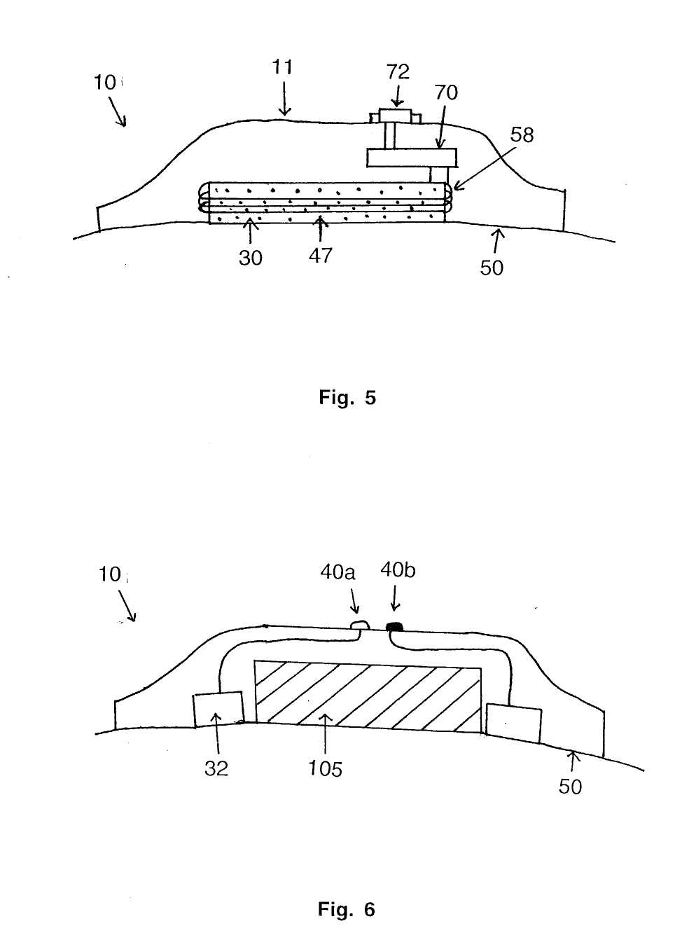

With continued reference to FIGS. 1B and 4C, FIG. 5 depicts a patch 10 comprising a fusion composition 30, placed proximate to the surface of the skin as in FIG. 4C, containing small conducting absorbing elements 47. The absorbing elements 47 are inductively heated by radiofrequency energy supplied to a coil 58 emplaced around the fusion composition 30. The battery 70 powers circuitry (not shown) that delivers the radiofrequency energy to the coil 58 and is modulated via a switch 72 connected to the battery 70. The switch 72 is located on the upper surface 11 of the patch 10.

FIG. 6 depicts a patch 10 comprising an annulus 32 in contact with the surface of the skin 50 and which is connected to terminals 40a,b. Emplaced within the area circumscribed by the annulus 32 is a material or medicament 105 in contact with the surface of the skin 50.

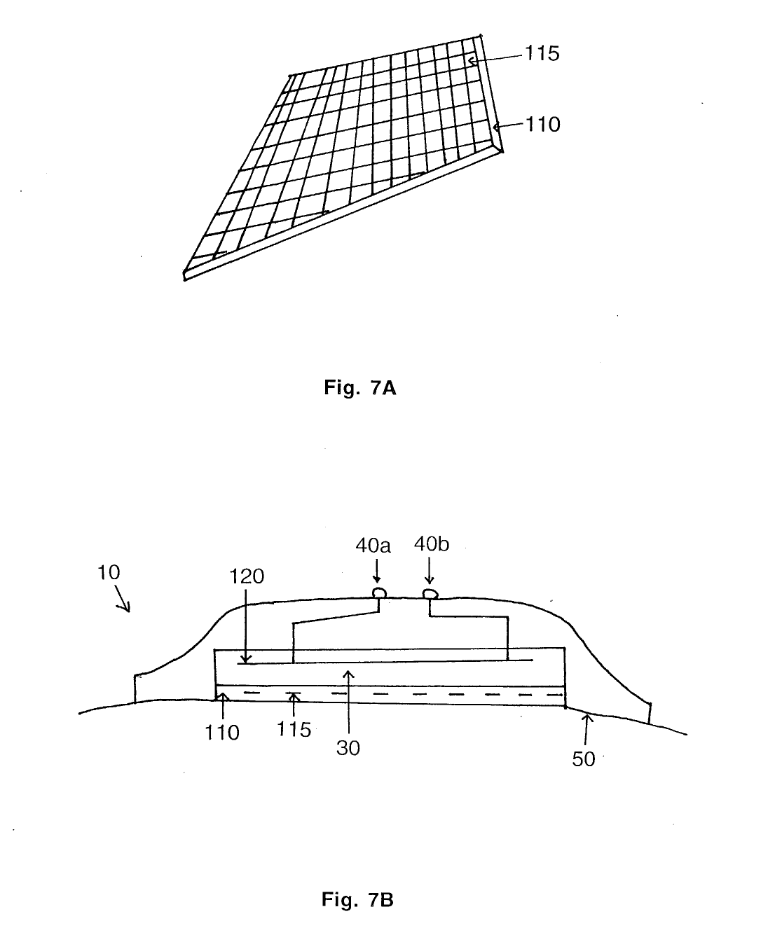

FIG. 7A depicts a fusion composition 110 having an arbitrary shape and capable of being cut with scissors or other sharp instrument. The fusion composition 110 incorporates an array of fine conducting elements 115. As shown in FIG. 7B, the fusion composition 110, cut in a desired shape, is contained within the patch 10 and placed over a wound on the surface of the skin 50. Material 30 which may be composed of a semisolid matrix containing 120 is placed over the fusion composition 110 and 120 is connected to exposed terminals 40a,b. The element 120 either conductively or inductively heats the fusion composition 110 via application of radiofrequency energy to terminals 40a,b which thus effects a weld at the skin 50.

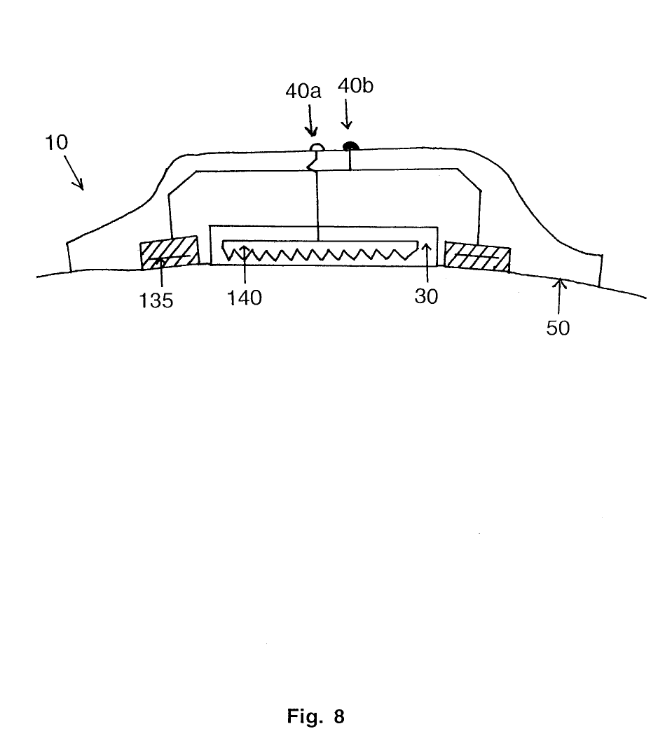

FIG. 8 depicts a patch 10 containing a fusion composition 30 placed on the skin 50. The fusion composition 30 contains an array of microneedles 140 proximate to the skin 50 which are connected to terminals 40a,b. An annular electrode 135 incorporating an electrically conductive fluid (not shown) also is connected to terminals 40a,b. Radiofrequency energy or a brief pulse or bipolar pulse of direct current through terminals 40a,b results in both tissue alterations of the skin 50 and thermal changes to the fusion composition 30.

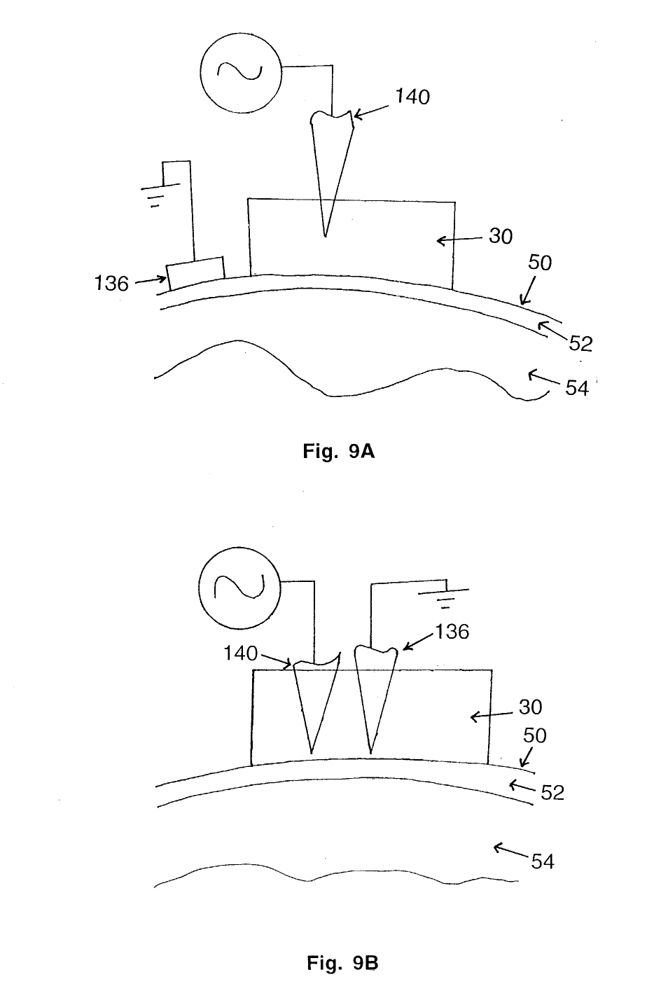

FIG. 9A depicts an active electrode 140 in contact with the fusion composition 30 which is placed on the stratum corneum 52 of the skin 50. A ground electrode 136 is located distal to the active electrode 140 and the fusion composition 30 and also is in contact with the stratum corneum 52. A plasma (not shown) formed, upon the application of radiofrequency energy or direct current, between the electrodes 140, 136 alters the stratum corneum without harming the epidermis 54 underneath the stratum corneum 52. Additionally, beneficial thermal changes are created within the fusion composition 30. Alternatively, FIG. 9B places both the active electrode 140 and the ground electrode 136 within the fusion composition 30.

FIG. 11 depicts an applicator 205 having an essentially solenoid coil structure 200 which is formed with an interior cylindrical zone 210. The solenoid coil 200 has electrical connectors 215a,b. The magnetic field lines 220 produced when an electrical current is passed through the electrical connectors 215a,b is shown. While the greatest magnetic intensity H (A/m) occurs within the applicator, a weaker magnetic field occurs at the ends and outside of the solenoid 200.

FIG. 12 depicts a clamp-like instrument 230 with which to apply an external oscillating magnetic field. The instrument 230 comprises a scissors-like extension having two arms 235a,b pivotally connected at the center 240. The arms 235a,b have a first end 245a,b attached to a coil 250a,b and have a second end 255a,b comprising a gripping means. The coils 250a,b form an essentially planar structure each having an outer surface 260a,b and an inner surface 265a,b and are each attached to a first end 245a,b of the arms 235a,b so that the inner surfaces 265a,b of the coils 250a,b are juxtaposed essentially horizontally and in parallel to each other. The pivotal action of the arms 235a,b increases or decreases the distance between the inner surfaces 265a,b of the inductive coils 250a,b such that the coils 250a,b may be positioned around tissue and/or other materials to effect bonding or fusing. The inductive coils 250a,b are attached to a radiofrequency source (not shown).

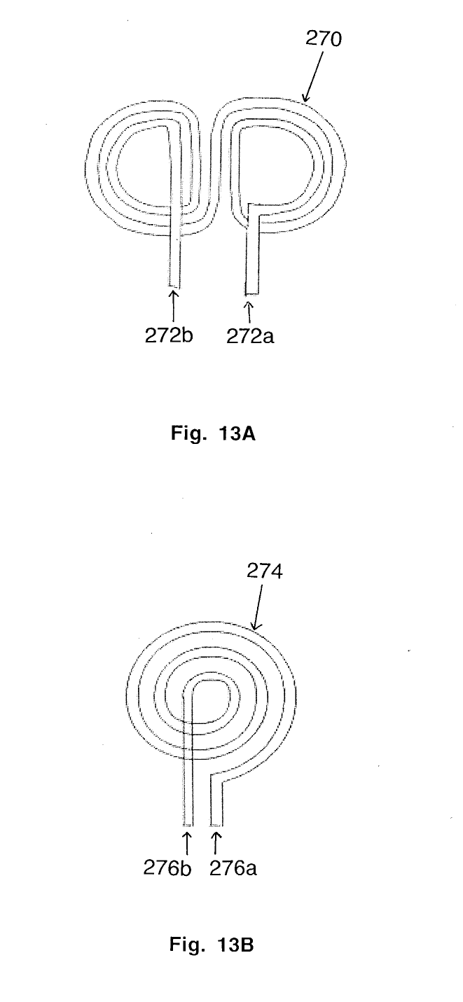

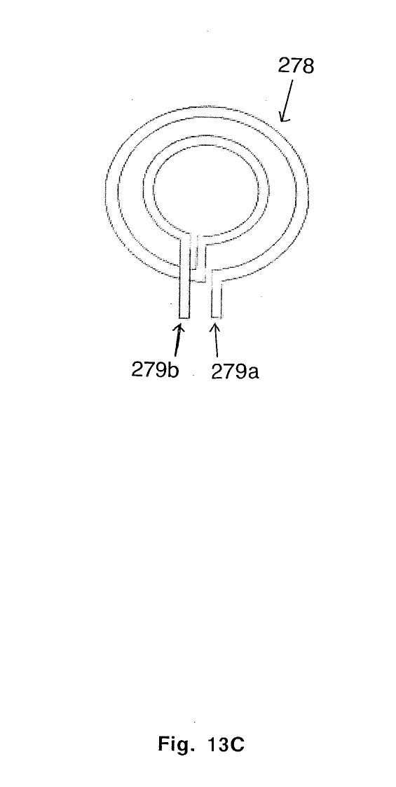

FIGS. 13A-13C depict substantially flat applicator coils for activating in other anatomical geometries. FIG. 13A shows a “butterfly coil” 270 with electrical connectors 272a,b. FIGS. 13B-13C show a spiral coil 274 with electrical connectors 276a,b and spiral coil 278 with electrical connectors 279a,b, respectively. Each coil 270,274,278 produces a magnetic field with a particular geometric shape. Coil 270 produces a two-lobed shaped field above and below the flat plane of the coil (not shown). With the addition of a material, such as mumetal, it is possible to shield the superior surface of the coil if no magnetic field is desired above the coil.

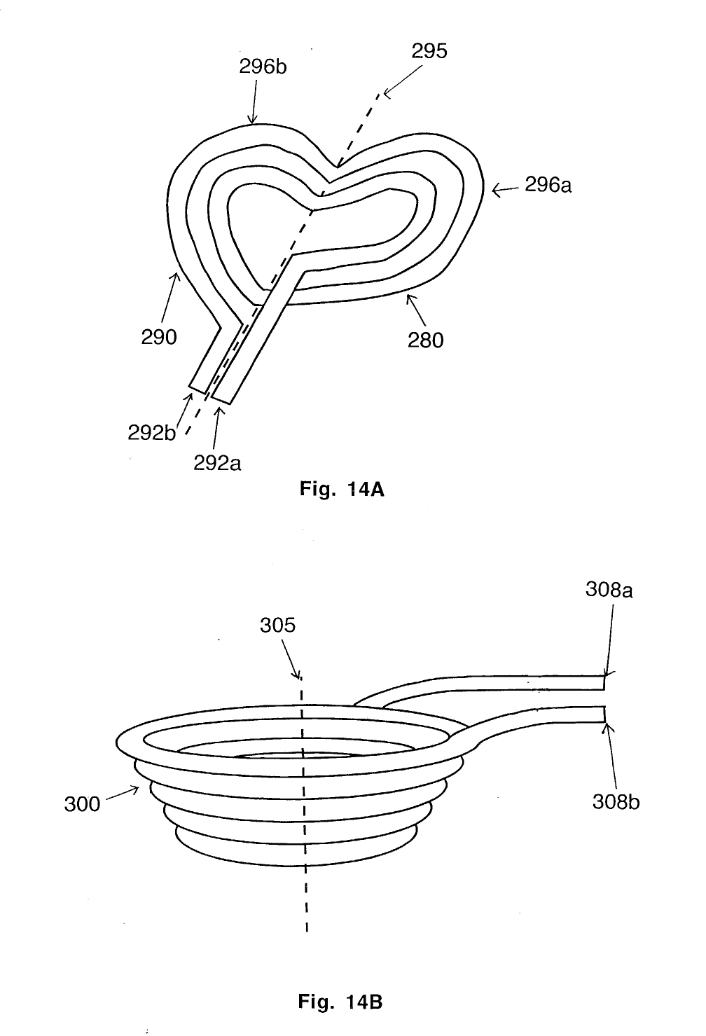

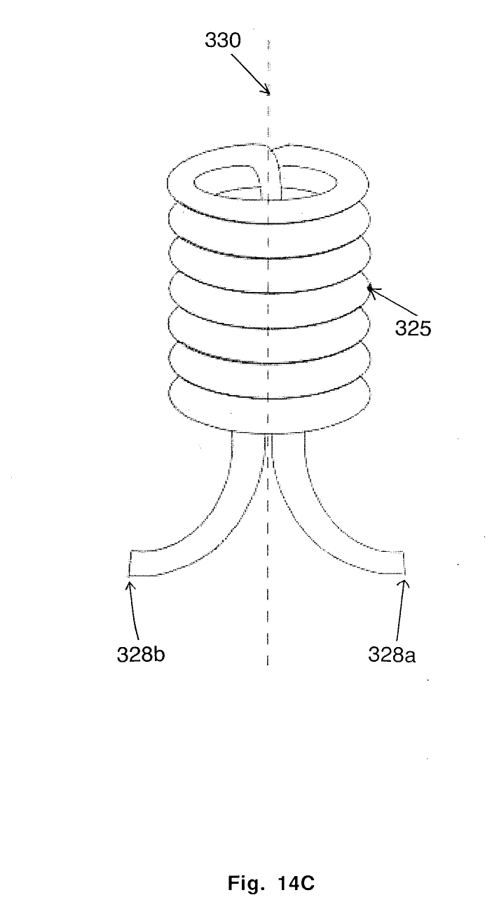

In FIGS. 14A-C and with continued reference to FIG. 13B, a non-planar coil applicator 280 is illustrated. The coil 290 with electrical connectors 292a,b is similar to coil 274 in FIG. 13B, but each half 296a,296b of coil 290 is bent towards the centerline 295, thus increasing the magnetic field intensity H at a position within a volume contained within the bent coil 290. FIG. 14B depicts a coil 300 with electrical connectors 308a,b which is in the form of a conical spiral with axis of symmetry 305. FIG. 14C shows a fusion applicator coil 325 with electrical connectors 328a,b which is symmetrical around axis 330 and which is designed for use in a hollow anatomical structure, such as a blood vessel (not shown).

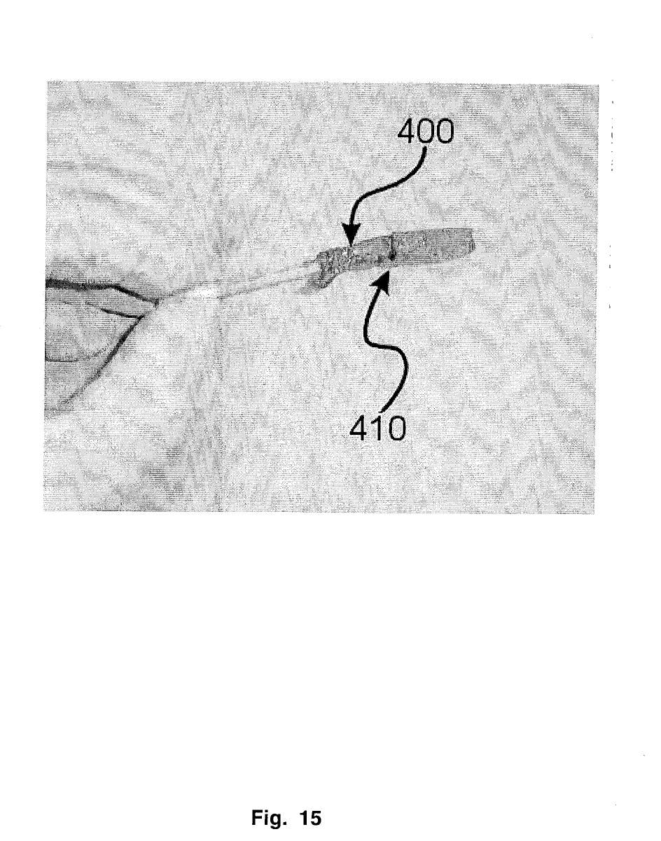

FIG. 15 depicts the visible fusion 410 of a vascular vessel 400.

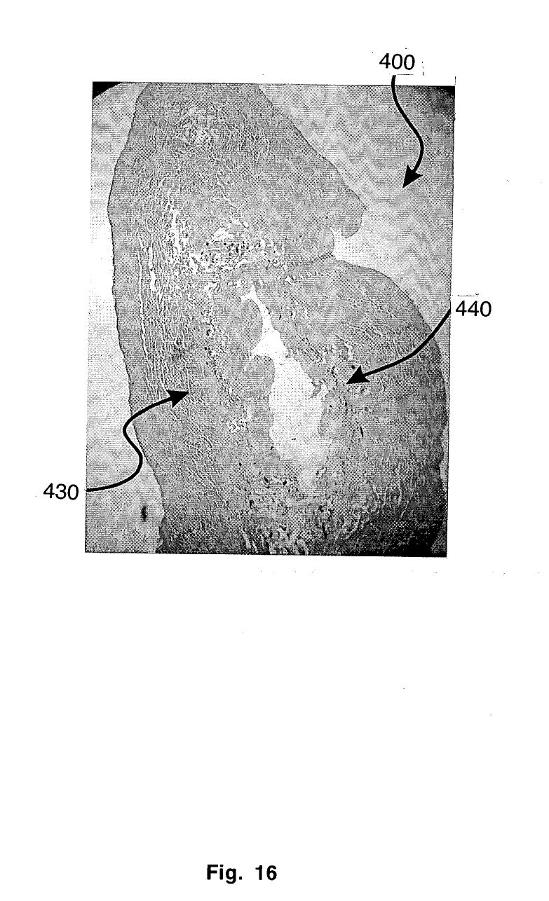

FIG. 16, with reference to FIG. 15, shows a histological section of the vascular vessel 400 with metallic particles 430 and 440 at the interface 410 between the two overlapping sections.

The following examples are given for the purpose of illustrating various embodiments of the invention and are not meant to limit the present invention in any fashion.

### EXAMPLE 1

Heating of Test Metal

The tissue fusion activator device constructed operates at a frequency of about 650 kHz and has an output of approximately 210 W. At or near this frequency, the skin depth in tissue for canine skeletal muscle at 1 MHz (10) is about 205 cm, while for nickel it is 160 μm. Thus, no significant heating of tissue occurs as a direct result of the field. Heating only occurs in close proximity to the fusion composition. Two solenoid-type applicator designs were used, and were made up of 200 turns of solid copper wire, 32 and 22 G, thus resulting in a coil approximately 2.86 cm in diameter and 0.95 cm in width. The bore of the coil was about 0.5 cm. The coils were encapsulated in a Pyrex sleeve, through which low-viscosity mineral oil (Sigma-Aldrich Inc., St. Louis, Mo.) was circulated as a coolant. In each of these coils, the magnetic intensity at the center of the coil is calculated to be greater than 10,000 A/m, while at approximately 0.5 cm from a single coil face the intensity is calculated to be maximally 160 A/m.

The blade of a small screwdriver (Craftsman Model 41541, 3.15 mm diameter) was positioned within the bore of the coils. After 1-5 seconds, the screwdriver was extracted and the blade was brought into brief contact with the skin of the hand. It was immediately apparent that significant heating had taken place in the blade of the screwdriver.

### EXAMPLE 2

Heating and Coagulating of Test Fusion Formulation

Fusion formulations were made of 50-75% (w/v) albumin (Bovine serum, or ovalbumin; Sigma-Aldrich, St. Louis, Mo.) in saline with a metal additive of 5% or 10% (w/v) nickel flake (average particle size=50 micron, Alfa Aesar, Ward Hill, Mass.) or 10% iron filings (particle size<30 microns; Edmund Scientific, Tonawanda, N.Y.)). Aliquots of approximately 1 ml of the fusion composition was positioned in thin-walled glass tubes with a diameter of about 4 mm. The tube was then positioned in the bore of the applicator. The device was energized for a period of 20-30 seconds. Evidence of denaturation and coagulation was ascertained visually as the material changed color. This was confirmed by probing the composition with a needle, which demonstrated evidence of increased viscosity or stiffness. The composition coagulated with all combinations of applicator and composition. Compositions with more metal or iron versus nickel heated at different rates.

### EXAMPLE 3

Fusion of Vascular Tissue

A series of experiments were performed using donated carotid, femoral and brachial artery samples harvested from sheep. The samples were rinsed in physiologic saline, placed in wet gauze, and frozen at −20° C. before use. After thawing, each sample was bisected lengthwise with a scalpel. The fusion formulation of 5% Ni and 50% albumin was placed around the periphery of one end of a bisected sample, i.e. on the adventitia, and the end of the other bisected sample was manually dilated and pulled over the fusion formulation so that there was an overlap of a few millimeters. A glass rod was positioned within the intima of the two vessels as a support to hold the tissue in place. The sample was then positioned between the faces of two opposing solenoid-type applicators, and the sample exposed to approximately 210 W of power for about 30 seconds.

As seen in FIG. 15, fusion of the vessel 400 was visually apparent 410, and the fused tissue could not be teased apart with forceps without damage to the tissue. There was no visual evidence of burning. Tests were repeated five times with equivalent results. The vessels were placed in 10% formalin, sectioned transversely, or perpendicular to the long-axis of the vessel, across the fused area and submitted for histological preparation and staining with hematoxylin-eosin. A sample histologic section is presented in FIG. 16 which shows the vessel 400 and the presence of metallic particles 430 and 440 at the interface between the two overlapping sections.

The following references are cited herein:

1. Bass, et al., Laser Surg. Med. 17, 315-349 (1995).

2. Freid, et al., Lasers Surg. Med. 27, 55-65 (2000).

3. Davies E J. Conduction and Induction Heating. Inst. Elect. Engs. and P. Peregrinus:London (1990).

4. Orfeuil M. Electric Process Heating: Technologies/Equipment/Applications. Battelle Press: Columbus Ohio (1987).

5. Zinn S. and Semiatin S L. Elements of Induction Heating—Design, Control and Applications, Electric Power Research Institute: Palo Alto, Calif. (1988).

6. Stauffer et al., IEEE Trans. Biomed. Eng. BME-31, 235-251 (1984).

7. Jordan A. et al, Int. J. Hyperthermia. 13(6):587-605 (1997).

8. Hamad-Schifferli et al., Nature 415, 152-155 (2002).

9. Damodaran S. Int. J. Biologic. Macromolec. 11, pp.2-8 (1989).

10. Francis Duck. Physical Properties of Tissue—A Comprehensive Reference Book. Academic Press: NY (1990).

Any patents or publications mentioned in this specification are indicative of the levels of those skilled in the art to which the invention pertains. These patents and publications are herein incorporated by reference to the same extent as if each individual publication was indicated as being incorporated specifically and individually by reference.

One skilled in the art will readily appreciate that the present invention is well adapted to carry out the objects and obtain the ends mentioned. It will be apparent to those skilled in the art that various modifications can be made in practicing the present invention without departing from the spirit or scope of the invention. Changes therein and other uses will occur to those skilled in the art which are encompassed within the spirit of the invention as defined by the scope of the claims.

What is claimed is:

1. A device to effect fusion between a tissue and at least one substrate to treat the tissue in an individual comprising:
a fusion composition;
a means to deliver a high frequency voltage or current to effect fusion; and
a means to control the extent of fusion.

2. The device of claim 1, wherein said fusion composition comprises a protein, a ferromagnetic material, a pharmaceutical, a conducting polymer, an ionic solution or combinations thereof.

3. The device of claim 2, wherein said protein comprises elastin, fibrin, collagen, albumin or combinations thereof.

4. The device of claim 1, wherein said polymer comprises a hydrogel, sol-gel, a synthetic biopolymer or combinations thereof.

5. The device of claim 1, further comprising a conductive material.

6. The device of claim 5, wherein said conductive material comprises a metal, a protein, a ferromagnetic material, a pharmaceutical, a conducting polymer, an ionic solution or combinations thereof.

7. The device of claim 5, wherein said conductive material is separate from but proximate to said fusion composition.

8. The device of claim 5, wherein said conductive material is embedded within said fusion composition.

9. The device of claim 1, wherein said means to deliver a high frequency voltage or current is an active terminal, a battery, or an active electrode, a ground electrode or combinations thereof.

10. The device of claim 9, wherein said active electrode and said ground electrode are embedded within said fusion composition.

11. The device of claim 10, wherein said active electrode, said ground electrode and said fusion composition comprise a dressing.

12. The device of claim 9, wherein said active electrode is embedded within said fusion composition and said ground electrode is distal to and external to said fusion composition.

13. The device of claim 8, wherein said active electrode is an electrode array, said electrode array comprising a plurality of isolated electrode terminals.

14. The device of claim 1, wherein said means to control the extent of said weld is electronic, a means to monitor the thermal history of the device or a means to detect changes in a ferromagnetic material comprising said fusion composition.

15. The device of claim 1, wherein said device is contained within a patch.

16. The device of claim 1, wherein said substrate is a tissue, a dressing or a fastening device.

17. A method of treating tissue in an individual by effecting a weld between the tissue and at least one substrate, comprising the steps of:
a) placing the device of claim 1 on the tissue of said individual;
b) delivering said high frequency voltage or current to said fusion composition comprising the device; and
c) monitoring the device to control the extent of the weld between said tissue and said substrate(s).

18. The method of claim 17, wherein steps b) and c) are repeated at least once.

19. A device to effect a weld between a tissue and a substrate to treat the tissue comprising:
a fusion composition or a conductive material or a combination thereof;
a means to inductively generate heat to effect the weld, and
a means to control the extent of the weld.

20. The device of claim 19, wherein said fusion composition and said conductive material independently comprise a protein, a ferromagnetic material, a pharmaceutical, a conducting polymer, an ionic solution or combinations thereof, said conductive material further comprising a metal.

21. The device of claim 20, wherein said protein comprises elastin, fibrin, collagen, albumin or combinations thereof.

22. The device of claim 20, wherein said conducting polymer comprises a hydrogel, sol-gel, a synthetic biopolymer or combinations thereof.

23. The device of claim 19, wherein said conductive material is separate from but proximate to said fusion composition.

24. The device of claim 19, wherein said conductive material is embedded within said fusion composition.

25. The device of claim 19, wherein the means to inductively heat generate heat comprises an induction coil to receive radiofrequency energy, said induction coil proximate to the device.

26. The device of claim 25, wherein said induction coil receives radiofrequency energy via circuitry comprising a battery and a switch.

27. The device of claim 25, further comprising a clamp-like instrument, said instrument comprising:
a first arm and a second arm, said first and second arms pivotally connected at the center, said first and second arms having a first end attached to said induction coils and a second end for manipulating and placing said induction coils proximate to said fusion composition and/or to said conductive material.

28. The device of claim 25, wherein said induction coil further comprises a coating of a smooth non-adhering material.

29. The device of claim 28, wherein said non-adhering material comprises teflon, titanium or gold.

30. The device of claim 19, wherein said means to inductively generate heat comprises a feedback control circuit to monitor voltage, current, impedence or magnetic field.

31. The device of claim 19, wherein said means to control the extent of said weld is electronic, a means to monitor the thermal history of the device or a means to detect changes in a ferromagnetic material comprising said fusion composition.

32. The device of claim 19, wherein said device is contained within a patch.

33. The device of claim 19, wherein said substrate is a tissue, a dressing or a fastening device.

34. A method of treating tissue in an individual to effect a weld between the tissue and at least one substrate, comprising the steps of:
a) placing the device of claim 19 on the tissue of said individual;
b) inductively heating said fusion composition or said conductive material comprising the device or a combination thereof to effect the weld; and
c) monitoring the device to control the extent of the weld between said tissue and said substrate(s).

35. The method of claim 34, wherein steps b) and c) are repeated at least once.

36. A device to heat biological materials comprising:
a fusion composition or a conductive material or a combination thereof; and
a means to inductively generate heat to effect heating of the biological materials.

37. The device of claim 36, wherein said fusion composition and said conductive material independently comprise a protein, a ferromagnetic material, a pharmaceutical, a conducting polymer, an ionic solution or combinations thereof, said conductive material further comprising a metal.

38. The device of claim 37, wherein said protein comprises elastin, fibrin, collagen, albumin or combinations thereof.

39. The device of claim 37, wherein said conducting polymer comprises a hydrogel, sol-gel, a synthetic biopolymer or combinations thereof.

40. The device of claim 36, wherein said conductive material is separate from but proximate to said fusion composition.

41. The device of claim 36, wherein said conductive material is embedded within said fusion composition.

42. The device of claim 36, wherein the means to inductively generate heat comprises an induction coil to receive radiofrequency energy, said induction coil proximate to the device.

43. The device of claim 42, wherein said induction coil receives radiofrequency energy via circuitry comprising a battery and a switch.

44. The device of claim 42, further comprising a clamp-like instrument, said instrument comprising:
a first arm and a second arm, said first and second arms pivotally connected at the center, said first and second arms having a first end attached to said induction coils and a second end for manipulating and placing said induction coils proximate to said fusion composition and/or to said conductive material.

45. The device of claim 42, wherein said induction coil further comprises a coating of a smooth non-adhering material.

46. The device of claim 45, wherein said non-adhering material comprises teflon, titanium or gold.

47. The device of claim 36, wherein said means to inductively generate heat comprises a feedback control circuit to monitor voltage, current, impedence or magnetic field.

48. The device of claim 36, further comprising means to control the extent of heating.

49. The device of claim 48, wherein said means to control the extent of heating is electronic, a means to monitor the thermal history of the device or a means to detect changes in a ferromagnetic material comprising said fusion composition.

50. The device of claim 36, wherein said biological materials comprise a tissue.

51. A method of heating biological materials, comprising the steps of:
a) placing the device of claim 36 proximal to the biological material; and
b) inductively heating said fusion composition or said conductive material comprising the device or a combination thereof to effect heating of the biological materials, said step optionally repeated at least once.

52. The method of claim 51, further comprising the step of:
c) monitoring the device to control the extent of the heating, said step optionally repeated at least once.
